(19) United States
(12) Patent Application Publication
(10) Pub. No.: US 2005/0014276 A1
(43) Pub. Date: Jan. 20, 2005

(54) Method for quatitative determination of dicarbonyl compounds

(76) Inventors: Wolfgang Kleiboehmer, Muenster (DE); Goeran Key, Osnabrueck (DE); Harald Peyrer, Emsdetten (DE); Uta Schulze-Pellengahr, Ascheberg (DE); Helga Korte, Muenchen (DE); Sabine Schreiber-Pietrek, Ascheberg (DE)

Correspondence Address: D Edward Dolgorukov;Marshall & Melhorn, 8th Floor, Four Seagate, Toledo, OH, 43604, US

(21) Appl. No.: 10/483,944

(22) PCT Filed: Jul. 9, 2002
(86) PCT No.: PCT/EP02/07623

(30) Foreign Application Priority Data
Jul. 16, 2001 (DE) 101 34 568.2

(51) Int. Cl. G01N 33/20; G01N 33/00

(52) U.S. Cl. 436/73; 436/128

(57) ABSTRACT

The present invention relates to a method for quantitative detrtmintaion of dicarbonyl compounds in gaseous and/or liquid samples and also aerosols. The method can hereby be used also for determination of the accumulation of reactive carbonyl compounds in the case of so-called carbonyl stress in diabetics.

The present invention relates to a method for quantitative determination of dicarbonyl compounds in gaseous and/or liquid samples and also aerosols. The method can hereby be used also for determination of the accumulation of reactive carbonyl compounds in the case of so-called carbonyl stress in diabetics.

Complications occurring as a result of long-standing diabetes diseases (insulin-dependent and/or insulin non-dependent diabetes), such as kidney damage or clouding of the eye lenses, can be detected only with difficulty and only belatedly. Corresponding markers for these diseases have to date only been possible in the laboratory via a complex blood analysis. Jointly responsible for these complications or consequential diseases are reactive metabolic products which, with collagen, enzymes and other cellular components, form glycolates and can act as cellular toxins due to this mechanism. In this connection, the role of α-oxoaldehydes, such as methylglyoxal, glyoxal and 3-deoxyglucuron, is discussed in particular. These materials are formed in the red blood corpuscles and occur in very low concentrations in blood plasma. In this way, they are also conveyed through the lung. In the lung, due to their membrane permeability they pass by diffusion via the alveolar membrane from the blood into the respiratory air. Hence, instead of in the blood, they can also be determined in respiratory air or in respiratory condensate.

The α-oxoaldehyde methylglyoxal is hereby of particular interest. Methylglyoxal can be produced from triose phosphate, from the metabolisation of ketone bodies and also in the case of metabolisation of threonine and is further degraded by glyoxalase. Methylglyoxal then reacts with proteins by forming imidazolone derivatives and bis-lysyl cross-links. This cross-linking of the proteins can lead to stabilisation of collagen and hence to thickening of membranes. Hence, these reactions explain at least a part of diabetic complications, such as kidney damage and lens clouding.

Blood sugar determination alone, as has been normal to date for diabetics, offers no direct conclusion about the momentary methylglyoxal concentration in whole blood, in plasma or in serum.

According to the state of the art, the determination of α-oxoaldehydes is therefore effected invasively, i.e. from blood plasma, apart from one exception. In the case of patients with insulin-dependent diabetes, there was able to be detected by Thornally T. J., “Advanced Glycation and the development of diabetic complications/Unifying the involvement of Glucose, methylglyoxal and oxidative stress”, Endocrinology and Metabolism, 1996, 3, 149-166, a five to six times higher methylglyoxal concentration in comparison to healthy comparative experimentees and, in the case of patients with insulin-dependent diabetes, a two to three times higher methylglyoxal concentration directly in the plasma. Alternatively, a determination of α-oxoaldehyde in the urine would be possible but is not described in the literature since the physiologically conditioned very low concentrations to be expected in the urine would demand too high an analytical complexity. A direct determination of the α-oxoaldehydes in the plasma is therefore required.

These methods according to the state of the art are consequently extremely complex and expensive and therefore have not been suitable to date for a regular routine check for determining the α-oxoaldehyde content in the whole blood, blood plasma or serum.

As a result of these examination methods according to the state of the art, regular checks and a possible needs-orientated dosing of medicines for targeted reduction of α-oxoaldehydes, such as methylglyoxal, in the whole blood, blood plasma and serum, are very complex.

The determination by means of gas chromatographic analysis methods is known from DE 100 28 326 A1. The increased AGE-chemistry in the body is however not restricted only to diabetes alone and is not based exclusively on oxidative processes. Increased substrate availability and reduced detoxification in the case of illness and with age likewise result in the accumulation of reactive carbonyl compounds (so-called carbonyl stress). In addition to the occurence of many diabetic complications, such as retinopathy, neuropathy and nephropathy, likewise an influence on the immune system by methylglyoxal-modified proteins was able to be observed, which by bonding to monocytes cause the distribution of proinflammatory cytokines. Furthermore, it is suspected that methylglyoxal plays a role in the pathogenesis of Alzheimer's syndrome and of vascular diseases. This opens up even further usage fields for respiratory air or respiratory condensate diagnostics.

Regular checking and possible needs-orientated dosing of medicines for targeted reduction of methylglyoxal has to date been very complex. The blood sugar determination alone, as is normal with diabetics, offers no direct conclusion about the momentary methylglyoxal concentration in the plasma.

Starting herefrom, it was the object of the present invention to provide a method for quantitative determination of dicarbonyl compounds, with which the determination of this compound in gaseous and/or liquid samples is made possible in a simple and cost-efficient manner.

This object is achieved by the generic method with the characterising features of claim 1. The further sub-claims show advantageous developments. The use of the method is described in claim 18.

According to the invention, a method for quantitative determination of dicarbonyl compounds in gaseous and/or liquid samples and also aerosols is provided, which comprises the following steps:
a) enrichment of the dicarbonyl compounds contained in the sample on a solid phase and/or on a suitable water-soluble polymer, which are derivatised with carbonyl-reactive groups, for example a hydrazide group. The result hereby is a coupling reaction between at least one carbonyl function of the dicarbonyl compound and the carbonyl-reactive group of the derivatised solid phase or of a suitable water-soluble polymer,
b) a coupling reaction between the dicarbonyl compound, and/or the immobilised compound, which is formed during the enrichment, with a specific analytical reagent,
c) a quantitative determination of the dicarbonyl compounds by means of the detection of the bonded analytical reagent with the method which is current according to the state of the art.

In a preferred embodiment, the method is implemented by means of microtitration plates. Both commercial microtitration plates which are suitable for the coupling of dicarbonyl compounds can thereby be used and a derivatisation of the microtitration plate surface can be effected, so that the desired functionality for bonding the dicarbonyl compounds is obtained.

Alternatively, the method can also be implemented by means of a flow injection system (English: flow injection analysis, FIA). The injection of the sample and of the further reagents is effected hereby into a carrier stream which is conveyed by means of peristaltic pumping. Likewise, other miniaturised through-flow systems are however also possible.

As a further different alternative, the method can also be implemented by means of a cartridge. The sample is conducted for this purpose by a cartridge which contains the solid phase, e.g. by means of a syringe. The dicarbonyl compounds in the cartridge are thereby bonded on the solid phase and can be subsequently be detected either internally or externally. The solid phase can occur thereby both as porous material or also in particle form.

Preferably, in step a), the coupling reaction between a first carbonyl function and the carbonyl-reactive group is implemented, and subsequently in step b), a coupling reaction between an analytical reagent and the second not yet bonded carbonyl function of the carbonyl compound. In step b), a fluorophoric hydrazide is thereby used as analytical reagent, which is detected subsequently in step c) fluorometrically. As fluorophoric hydrazides, there are possible for example the following compounds:
a) 5-(((2 -carbohydrazino)methyl) thio)acetyl)aminofluorescein;
b) fluorescein-5-thiosemicarbizide;
c) 4,4-difluoro5,7dimethyl-4-bora-3a,4a-diaza-s-indacene-3-propionic acid-hydrazide;
d) Texas red® 350;−488;−568;−594;−633.

Alternatively, in step b), a compound which contains hydrazide groups can be used which subsequently is coupled with an antibody which is specific for this compound. This antibody can be marked preferably with an analytical reagent, e.g. a fluorophore, gold or latex particles.

As a further variant, the antibody can be marked with an enzyme as analytical molecule.

As an additional variant, the use of an avidin-enzyme complex as analytical reagent is presented in step b). In the use of this usage variant, a biotin hydrazide is bonded to the second carbonyl function of the dicarbonyl compound, an enzyme being able to be coupled via the biotin-avidin interaction. By using avidin, a signal amplification is possible due to its tetracovalency by cross-linking of the avidin-enzyme conjugates by a dibiotin. This cross-linking can be effected already in advance.

In the case of coupling of an enzyme, the detection of the same is effected preferably photometrically, fluorometrically, electrochemically and/or luminometrically in step c).

A further variant for the detection resides in a high molecular compound being coupled to the dicarbonyl compound and the latter subsequently being detected by means of the increase in mass. For this purpose, for example quartz microbalances or also direct-optical methods, such as surface plasmon resonance (English: surface plasmon resonance, SPR), are possible.

Likewise all the detection methods known from the state of the art can however also be used.

A further variant for the detection resides in using in step b) nano- or microparticles which are suitable for optical analysis and carry carbonyl-reactive groups on their surface. These groups react with the second carbonyl function of the dicarbonyl compound. The detection can be effected photometrically, fluorometrically, nephelometrically, turbidimetrically or visually.

In a further variant, a coupling reaction between the immobilised compound, which is formed during the enrichment, with a specific analytical reagent can be effected in step b). Preferably compounds from the group aminopyrazines, aminopyridines, aminopyrimidines and aminoguanidines are thereby selected as carbonyl-reactive groups. These compounds are able to bond covalently the carbonyl compounds, such as methylglyoxal or glyoxal, via their two carbonyl groups. In the case of this variant, these compounds are therefore immobilised on the solid phase so that they serve subsequently as collector component for the dicarbonyl compounds. The component formed due to the reaction between the dicarbonyl compound and the said compounds can be detected subsequently by means of an antibody which is specific for this component. This antibody is however not reactive with the free dicarbonyl compounds or the collector component. The antibody can be marked with an analytical molecule, for which for example fluorophores, enzymes, latex or gold particles are possible. It is likewise also possible that the antibody is detected by means of an anti-antibody or via the avidin-biotin system.

A further alternative provides that, in step b), the solid phase or the water-soluble polymer, which are derivatised with carbonylreactive groups, serve as specific analytical reagent, an aromatic compound arising due to a ring closure reaction. These in turn can be detected in step c) photometrically or fluorometrically. The detectable product is produced in this variant by the two-step coupling reaction independently, without further coupling of an additional analytical reagent.

α-oxoaldehydes are determined as preferred dicarbonyl compound by means of the method. There are included herein for particular preference methylglyoxal, glyoxal and/or 3-deoxyglucuron.

The method is implemented in gaseous and also liquid samples and there should be mentioned by way of example respiratory air, respiratory condensate, sputum, bronchio-alveolar lavage, blood, plasma, serum, urine, tissue fluid and tear fluid.

The method according to the invention is intended to be explained in more detail by means of the two following Figures, without said method being limited hereto.

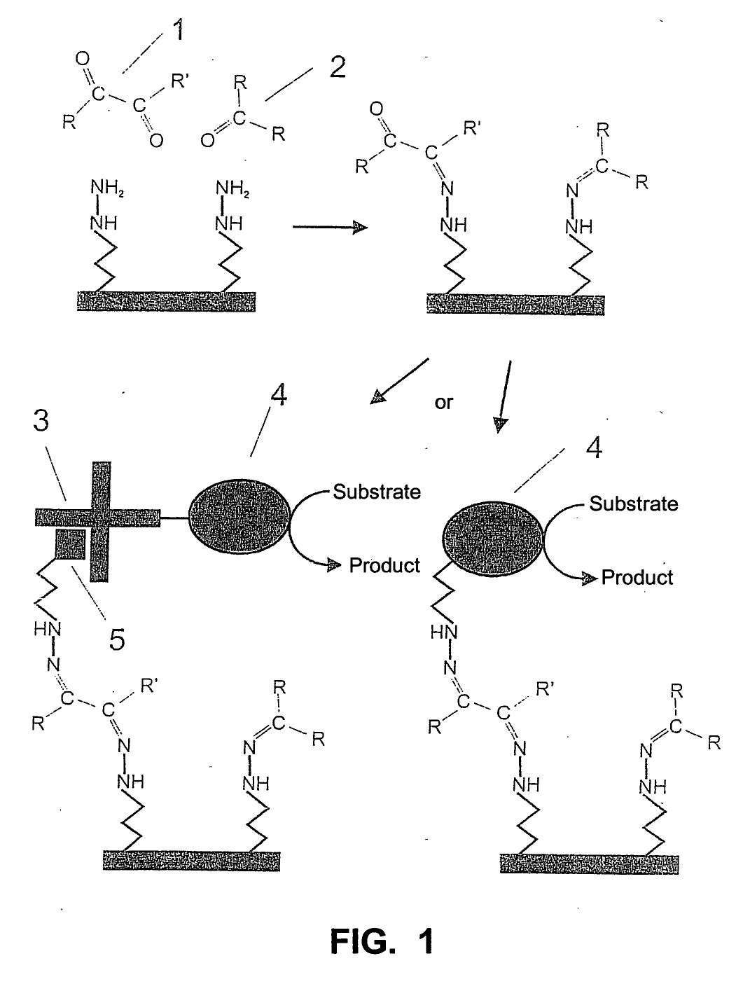

FIG. 1 shows two examples of the schematic course of the method according to the invention. Firstly, the coupling of the dicarbonyl compounds, here methylglyoxal (1), to the solid phase derivatised with hydrazide groups is effected.

Acetone (2) as monocarbonyl compound is likewise bonded to the solid phase but has no further free carbonyl function for the coupling of the analytical reagent, as a result of which the monocarbonyl compounds are not jointly detected and interference by the latter is avoided. The subsequent coupling of the enzyme is effected via two variants. In the first variant, the dicarbonyl compound reacts via the second, still free carbonyl function with a biotin (5) which is bonded to a hydrazide. In a further step, the biotin (5) is coupled to an avidin molecule (3), to which in turn an enzyme (4) is bonded.

In contrast, in the further variant, a direct bonding of an enzyme (4) which is modified with a hydrazide group is effected.

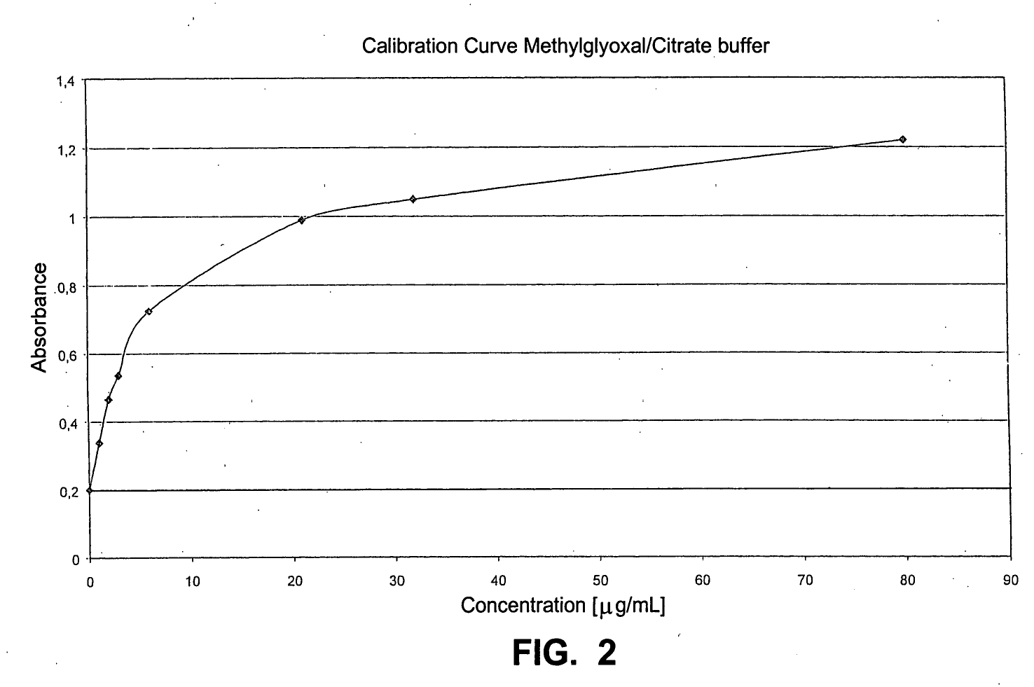

FIG. 2 shows the result of testing which is implemented according to the same principle, with detection via biotin hydrazide and avidin peroxidase with subsequent absorption measurement.

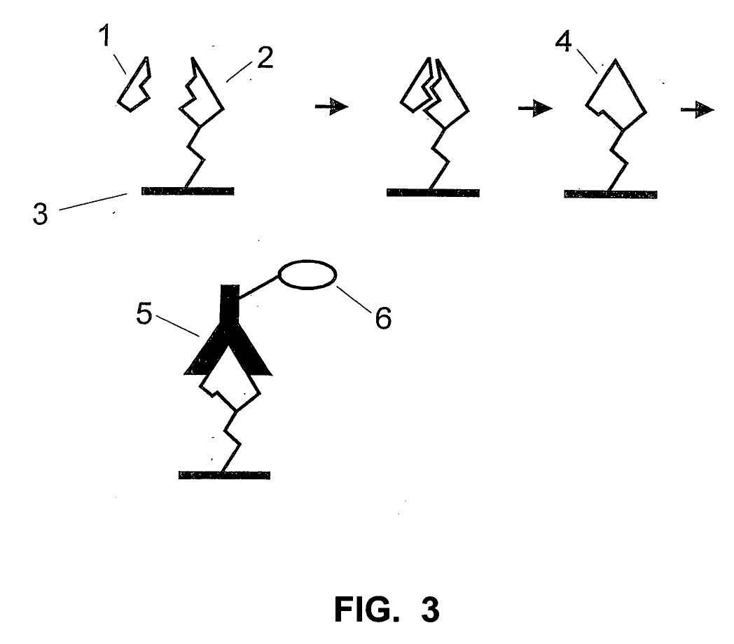

FIG. 3 shows the principle of detection of the component formed due to the reaction between dicarbonyl compound and the carbonyl-reactive collector component, by means of a specific antibody.

Methylglyoxal (1) reacts with the collector component (2), which is immobilised on the solid phase (3), to form a component (4) which is detected by a specific antibody (5) which is provided with a label (6).

1-20. (canceled).

21. A method for quantitative determination of dicarbonyl compounds in gaseous and/or liquid samples with the following steps:
a) enrichment of the dicarbonyl compounds contained in the sample on a solid phase and/or on a water-soluble polymer, which are derivatised with carbonyl-reactive groups, via a coupling reaction between at least one carbonyl function of the dicarbonyl compound and the carbonyl-reactive group,
b) coupling reaction of the dicarbonyl compound and/or of the immobilised compound, which is formed during the enrichment, with a specific analytical reagent, and
c) quantitative determination of the dicarbonyl compounds by means of the detection of the quantity of the bonded analytical reagent.

22. The method according to claim 21, wherein the method is implemented by means of microtitration plates.

23. The method according to claim 21, wherein the method is implemented in a flow injection system.

24. The method according to claim 21, wherein the method is implemented in a miniaturised through-flow system.

25. The method according to claim 23, wherein, in step a), the sample is conducted through a cartridge which contains the solid phase.

26. The method according to claim 25, wherein, in step a), the coupling reaction between a first carbonyl function and the carbonyl-reactive group is implemented, and subsequently in step b), a coupling reaction between the analytical reagent and the second carbonyl function of the dicarbonyl compound is implemented.

27. The method according to claim 26, wherein, in step b), a fluorophoric hydrazide is used as analytical reagent and this hydrazide is detected fluorometrically in step c).

28. The method according to claim 26, wherein, in step b), an avidin-enzyme complex is used as analytical reagent, which is bonded to the second carbonyl function of the dicarbonyl compound via a biotin hydrazide.

29. The method according to claim 26, wherein, in step b), nano- or microparticles are used, which are modified on the surface with carbonyl-reactive groups, and which are subsequently detected photometrically, fluorometrically, nephelometrically, turbidimetrically or visually.

30. The method according to claim 26, wherein, in step b), a compound which contains hydrazide groups is used, which compound is subsequently coupled with an antibody which is specific for this compound.

31. The method according to claim 30, wherein the antibody is marked with an analytical reagent, for example with a fluorophore, gold particles or latex particles.

32. The method according to claim 30, wherein the antibody is marked with an enzyme as analytical molecule.

33. The method according to claim 28, wherein, in step c), detection is implemented photometrically, fluorometrically, electrochemically, luminometrically, direct-optically and/or with a quartz microbalance.

34. The method according to claim 21, wherein the carbonyl-reactive group in step a) is selected from the group of aminopyrazines, aminopyridines, aminopyrimidines and aminoguanidines.

35. The method according to claim 34, wherein a marked antibody is used as analytical reagent which is specific for the immobilised compound.

36. The method according to claim 35, wherein the antibody is marked with a fluorophore, an enzyme, gold particles, a marked anti-antibody or an avidin-biotin system.

37. The method according to claim 21, wherein, in step b), the solid phase and/or the watersoluble polymer, which are derivatised with carbonyl-reactive groups, function as specific analytical reagent, an aromatic component being produced by a ring closure reaction, which component is detected in step c) photometrically or fluorometrically.

38. The method according to claim 21, wherein α-oxoaldehydes are determined as dicarbonyl compound.

39. The method according to claim 38, wherein methylglyoxal, glyoxal and/or 3-deoxyglucuron is determined as dicarbonyl compound.

40. Utilization of the method according to claim 21 for determination of dicarbonyl compounds in respiratory air, respiratory condensate, body fluids and/or bronchioalveolar lavage.
